(19) United States
(12) Patent Application Publication
(10) Pub. No.: US 2024/0198099 A1
(43) Pub. Date: Jun. 20, 2024

(54) COMPOSITIONS AND METHODS FOR TREATING CANCER-ASSOCIATED CACHEXIA

(71) Applicant: Duke University, Durham, NC (US)

(72) Inventors: Xiling SHEN, Durham, NC (US); Aliesha O'RAW, Durham, NC (US); James MORIZIO, Durham, NC (US); Ashlesha DESHMUKH, Durham, NC (US); Ayelet EREZ, Bnei Zion (IL); Saar EZAGOURI, Rehovot (IL)

(21) Appl. No.: 18/004,492

(22) PCT Filed: Jul. 9, 2021
(86) PCT No.: PCT/US21/41029

§ 371 Date: Jan. 6, 2023

Related U.S. Application Data
(60) Provisional application No. 63/050,352, filed on Jul. 10, 2020.

(51) Int. Cl. A61N 1/36; A61N 5/06

(52) U.S. Cl. CPC A61N 1/36002; A61N 1/36034; A61N 5/0622

(57) ABSTRACT

A method for treating cachexia in a subject in need thereof includes stimulating the parasympathetic nervous system of the subject thereby treating cachexia in the subject. Stimulating the parasympathetic nervous system can increase expression of urea cycle enzymes in the liver of the subject. Parasympathetic nervous system stimulation can comprise stimulating the vagus nerve, for example, the cervical vagus nerve or the hepatic branch of the vagus nerve. Pulses can be delivered at a frequency ranging from 1 Hz to 10 Hz or at a frequency of about 5 KHz.

### CROSS-REFERENCE TO RELATED APPLICATIONS

This application claims priority to U.S. Provisional Patent Application No. 63/050,352, filed on Jul. 10, 2020, which is incorporated by reference herein in its entirety.

### BACKGROUND

Cancer-associated cachexia (CAC) is a multifactorial syndrome defined by an ongoing loss of skeletal muscle mass, with or without loss of fat mass, that cannot be entirely reversed by conventional nutrition support. The onset of CAC increases chemotherapy toxicity and complications from surgeries, decreases quality of life in patients, and leads to higher mortality rates. In many instances, after onset of CAC in a patient, chemotherapy treatment is discontinued because of the inability of chemotherapy treatment to be effective in a patient who has developed CAC. Cachexia is prevalent among the deadliest cancers, contributes to half of all cancer deaths worldwide, is irreversible, and associates with end stage disease. Currently, there is no effective treatment available for CAC.

A key feature of CAC is chronic deterioration of lean body mass. Symptoms include weight loss, muscle wasting, nutrient deficiency, and loss of appetite. People who develop cachexia are not losing weight because they are trying to trim down with diet or exercise. Rather, they lose weight because they eat less due to a variety of reasons. At the same time, their metabolism changes, which causes their body to break down too much muscle.

In patients with cancer, tumor cells release substances that reduce appetite and cause the body to burn calories more quickly than usual. Cancer and its treatments can also cause severe nausea or damage the digestive track, making it difficult for cancer patients to eat and absorb nutrients. As the body gets fewer nutrients, it burns fat and muscle, and cancer cells use what limited nutrients are left to survive and multiply.

Previous attempts to treat cancer-associated cachexia focused on the symptoms such as muscle wasting and nutrient deficiency. However, clinical trials based on nutrient supplements and anti-inflammatory treatments have all failed primarily because the focus on the treatment has been on the symptom rather than the root cause—the patient's brain and metabolism processing. Hence, there is a need for new and improved therapies for the treatment of cancer-associated cachexia.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In a first aspect of the invention, a method for treating cachexia in a subject in need thereof comprises stimulating the parasympathetic nervous system of the subject thereby treating cachexia in the subject. In a feature of this aspect, stimulating the parasympathetic nervous system in the subject increases expression of urea cycle enzymes in the liver.

In additional aspects of the invention, methods for mitigating weight loss due to cachexia, mitigating fat loss due to cachexia, mitigating muscle wasting due to cachexia, and mitigating loss of appetite due to cachexia comprise stimulating the parasympathetic nervous system in the subject.

In another aspect of the invention, a method for mitigating urea cycle dysregulation in a subject in need thereof due to cachexia comprises stimulating the parasympathetic nervous system in the subject thereby mitigating urea cycle dysregulation due to cachexia in the subject.

In a further aspect of the invention, stimulating the parasympathetic nervous system comprises stimulating the vagus nerve. In a feature of this aspect, stimulating comprises stimulating the cervical vagus nerve or the hepatic branch of the vagus nerve. Stimulating the vagus nerve can comprise delivering pulses at a frequency ranging from 1 Hz to 10 Hz. Regarding this feature, the pulses can have a pulse width of about 1 millisecond to about 100 milliseconds. In another feature, stimulating pulses can be delivered at a frequency of about 5 kHz. Regarding this feature, the pulses can have a pulse width of greater than 0 and less than 0.2 milliseconds.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1 is a schematic diagram showing the autonomic activities of the sympathetic nervous system (SNS) and parasympathetic nervous system (PNS) during different physiological states.

FIG. 2A provides an exemplary schematic diagram of functional electrical stimulation parameters.

FIG. 2B provides an exemplary schematic diagram of stimulation pattern duration.

FIG. 3 is a graph showing the fold-changes of hepatic amino acid levels in cachectic mice using Student's t-test, Bonferroni FDR<0.05 in accordance with Example 1.

FIGS. 4A-3D are schematic illustrations and images showing exemplary denervation surgery.

FIGS. 5A-4D are images showing exemplary electrical stimulation of hepatic sympathetic and parasympathetic nerves.

FIG. 6 is a schematic illustration showing a block diagram outlining an exemplary stimulation and recording pipeline.

FIGS. 7A and 7B are graphs showing blood glucose level after sympathetic (Symp), parasympathetic (Para) and sham (No stim) stimulations.

FIGS. 8A-8C are graphs showing hepatic metabolic gene expression levels quantified by qRCR after sympathetic and parasympathetic stimulation.

FIG. 9 is a series of photographs (top and bottom) and a schematic illustration of experimental procedures for Example 4.

FIGS. 10A-10D are charts and graphs illustrating how body weight was affected by cancer injection and vagus nerve stimulation.

FIGS. 11A and 11B are charts showing the effect of VNS or the absence of VNS on total fat and brown adipose tissue, respectively.

FIG. 12A includes photographs of skeletal muscle fiber for Control (top) and Cancer (bottom) mice.

FIG. 12B is a chart comparing muscle atrophy for mice having cancer, cancer with VNS therapy, cancer with vagotomy, and healthy control.

FIG. 13 is a chart showing the effect of vagal nerve stimulation on daily food intake for control mice, mice with cancer but no treatment, and cancer with VNS treatment.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably and refer to both human and nonhuman animals. The term “nonhuman animals” includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human who is suffering from cancer associated cachexia (CAC).

Unless otherwise defined, all technical terms used herein have the same meaning as commonly understood by one of ordinary skill in the art to which this disclosure belongs.

Cachexia is a condition related to uncontrolled weight loss that occurs incidental to many severe diseases, including cancer, sepsis, and major organ failure. Cachexia is defined as the unwanted loss of a least 5% of lean mass within six months. Cachexia often represents the last step of a chronic disease. The condition affects many advanced cancer patients and is associated with poor prognosis regardless the tumor nature. It is generally accepted that cachexia is indirectly responsible for the death of at least 20% of all cancer patients. The incidence of cachexia among cancer patients is remarkably high, although it varies by tumor type. In patients with gastric or pancreatic cancer, the incidence is more than 80%, whereas approximately 50% of patients with lung, prostate or colon cancer are affected, and around 40% of patients with breast tumors or some leukemias develop the cachexia.

Loss of skeletal muscle mass is recognized as an independent predictor of mortality and is associated with functional impairment, altered quality of life and reduced tolerance and response to anticancer therapies. Further, it has been shown that reversal of muscle loss leads to prolonged survival in animal models of cancer cachexia. These observations support that maintaining muscle mass is helpful in improving survival in cachectic conditions. Understanding molecular drivers of cachexia is important for the developing management strategies.

Cachexia affects various organs, which often results in systemic complications. The molecular mechanisms of cancer cachexia are not well characterized. Generally, scientists believe that cachexia results from abnormal metabolism and anorexia. The development of muscle atrophy results from an imbalance between muscle protein synthesis and degradation inducing a decrease in myofibrillar and sarcoplasmic proteins illustrated by muscle fiber shrinkage. However, the nature of the key factors responsible for muscle atrophy in cancer cachexia is unknown.

The present disclosure is based, in part, on the discovery that manipulation of the vagus nerve can be effective in reversing and/or mitigating cachexia. In embodiments, manipulating can include stimulation or denervation. Moreover, in embodiments, stimulation can include, without limitation, electrical stimulation or optogenetic stimulation. Much of the description provided herein relates to electrical stimulation. However, one of ordinary skill in the art will understand that stimulation may comprise optogenetic stimulation. Manipulating the vagus nerve via vagus nerve stimulation or denervation targets the gut-brain axis and can effectively reverse and/or mitigate cachexia.

The gut-brain axis is a bidirectional link connecting the gut and the brain and including communication between the central nervous system and the enteric nervous system of the body. The gut-brain axis includes communication between the endocrine (hypothalamic-pituitary-adrenal axis), immune (cytokine and chemokines) and the autonomic nervous system (ANS). In animal studies related to the gut-brain axis, stress was seen to inhibit signals sent through the vagus nerve and cause gastrointestinal problems. Similarly, a study in humans found that people with irritable bowel syndrome (IBS) or Crohn's disease had reduced vagal tone, indicating a reduced function of the vagus nerve.

Described herein is a method of treating cachexia in a subject in need thereof. The method comprises stimulating the parasympathetic nervous system of the subject thereby treating cachexia in the subject. It is contemplated that stimulating the parasympathetic nervous system may increase expression of urea cycle enzymes in the liver thereby leading to reversal or mitigation of cachexia.

As mentioned previously, cachexia is marked by uncontrolled weight loss. Symptoms include muscle wasting, nutrient deficiency, and loss of appetite. Accordingly, described herein are methods of reversing or mitigating the symptoms of cachexia. In embodiments, this includes a method of mitigating weight loss due to cachexia in a subject in need thereof, wherein the method comprises stimulating the parasympathetic nervous system in the subject thereby mitigating weight loss due to cachexia. In embodiments of the method, the subject having cachexia who received stimulation to the parasympathetic nervous system experiences statistically significantly less weight loss than subjects having cachexia that received no stimulation. In additional embodiments, there is no statistically significant variation in weight loss between the subject having cachexia who received stimulation to the parasympathetic nervous system and a healthy control subject. Additionally, described herein is a method of mitigating fat loss due to cachexia in a subject in need thereof, wherein the method comprises stimulating the parasympathetic nervous system in the subject thereby mitigating fat loss due to cachexia. In embodiments, the fat is brown adipose tissue. Regarding this embodiment, there is no significant change in brown adipose tissue mass between healthy control subjects and the subject having cachexia who received stimulation to the parasympathetic nervous system. Moreover, regarding this embodiment, a subject having cachexia who received stimulation to the parasympathetic nervous system experiences statistically significantly less atrophy of brown adipose tissue than subjects having cachexia that received no stimulation. Additionally, methods of mitigating muscle wasting due to cachexia in a subject in need thereof are described. The method comprises stimulating the parasympathetic nervous system in the subject thereby mitigating muscle wasting due to cachexia. Additionally, methods of mitigating loss of appetite due to cachexia in a subject in need thereof are described. The methods comprise stimulating the parasympathetic nervous system in the subject thereby mitigating loss of appetite. In embodiments, there is no statistically significant variation in average daily food intake between subjects having cachexia who received stimulation to the parasympathetic nervous system and healthy control subjects. In alternative embodiments, subjects having cachexia who received stimulation to the parasympathetic nervous system had statistically significantly higher daily food intake than subjects having cachexia that received no stimulation.

Additionally, in embodiments, a method of reversing and/or mitigating cachectic urea cycle dysregulation in a subject in need thereof is described. The method comprises stimulating the parasympathetic nervous system in the subject thereby reversing and/or mitigating cachectic urea cycle dysregulation in the subject.

The urea cycle is the primary means of nitrogen metabolism in humans and other ureotelic organisms. There are five prominent hepatic enzymes in the urea cycle: carbamoyl-phosphate synthetase (CPS), ornithine transcarbamylase (OTC), argininosuccinate synthetase (ASS), argininosuccinase lyase (ASL), and arginase (ARG). In healthy individuals, muscle breakdown leads to the flow of amino acids into the liver, where excess nitrogen reacts with aspartate to synthesize urea via the urea cycle to be disposed by urine. Outside the liver, different urea cycle enzymes are expressed to provide urea cycle intermediates arginine and ornithine to supply cellular needs.

Without being bound by theory, it is believed that dysregulation of the expression of urea cycle (UC) enzymes promotes cancer proliferation by diversion of aspartate and glutamine toward pyrimidine rather than urea synthesis. In particular, it is believed that in various types of CAC, the expression and function of hepatic urea cycle enzymes is downregulated despite the high flux of amino acids that is generated by the protein breakdown of skeletal muscle secondary to an aberrant signaling cascade caused by cancer. This unexpected result suggests that dysregulated urea cycle enzyme expression in the liver of the host is part of the systemic dysregulation induced by the tumor to increase nitrogen availability for its needs. Experimental results provided below demonstrate that the overall increase in protein turnover measured in cachexia patients cannot be explained solely by tumor cellular turnover. This result explains previously unexplained systemic dysregulation in nitrogen homeostasis experienced by subjects who developed cachexia.

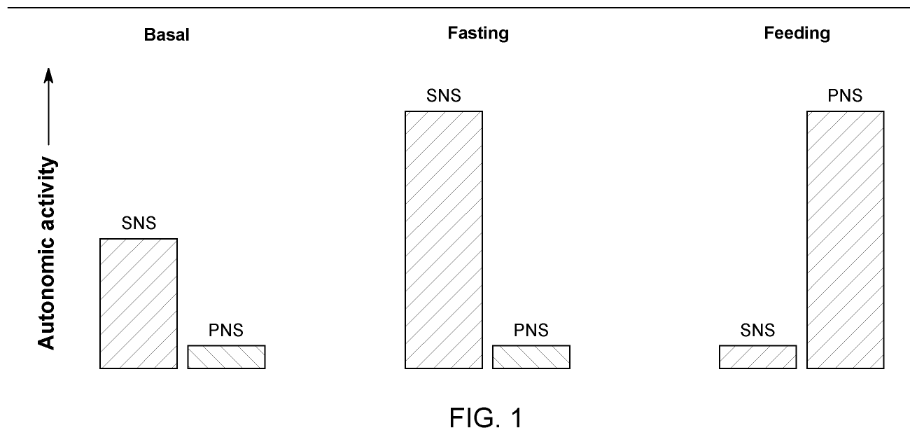

The autonomic nervous system (ANS) controls specific body processes, such as circulation of blood, digestion, breathing, urination, heartbeat, etc. The primary function of the autonomic nervous system is homeostasis. Apart from maintaining the body's internal environment, it is also involved in controlling and maintaining multiple life processes including digestion and metabolism. There are two types of autonomic nervous system: sympathetic autonomic nervous system and parasympathetic autonomic nervous system. The sympathetic autonomic nervous system is located near the thoracic and lumbar regions in the spinal cord. Its primary function is to stimulate the body's fight or flight response. The sympathetic nervous system is primarily associated with the energy mobilization and fasting phase of systemic metabolism. The parasympathetic autonomic nervous system is located between the spinal cord and the medulla. It primarily stimulates the body's “rest and digest” and “feed and breed” response. The parasympathetic nervous system is primarily associated with the feeding phase of systemic metabolism. The parasympathetic nervous system includes the parasympathetic vagus nerve. FIG. 1 is a bar graph illustrating some of the functionalities of the sympathetic and parasympathetic nervous systems.

Vagal nerve manipulation, in particular vagus nerve stimulation (VNS), is an FDA-approved therapy for treatment-resistant focal epilepsy, treatment-resistant major depressive disorder, episodic cluster headaches, and migraine pain. VNS is under additional investigation as a clinical tool for treatment of obesity, anxiety disorders, dementia, alcohol addiction, chronic heart failure, arrhythmia, autoimmune diseases, and chronic pain conditions. Moreover, studies have reported promising outcomes following vagus nerve manipulation—both stimulation and blockade—for treatment of obesity-associated metabolic syndromes in clinical trial and preclinical studies. Additionally, vagus nerve stimulation, in which the nerve is stimulated with pulses of electricity, has been used to treat patients with epilepsy, depression, Alzheimer disease and migraine.

VNS is also being investigated for treatment of inflammation in several autonomic or inflammatory disorders. Preliminary studies have evaluated VNS being used for stroke, autoimmune diseases, heart and lung failure, obesity, and pain management, but further studies are needed to understand the mechanistic actions that explain VNS's potential role in treating these disorders.

Despite any of the foregoing, prior to the work described herein, vagus nerve manipulation, including denervation and stimulation, had not been investigated to treat, reverse, or mitigate cachexia. Moreover, in clinical trials of anti-inflammatory therapies, such as TNF-a and interleukins, the therapies were shown to not benefit cachexic patients. Accordingly, it is unlikely that a VNS effect on cachexia is related to inflammation, or to inflammation alone. Rather, in view of the work described herein, the effect of VNS on cachexia is believed to be related to regulating the gut-brain axis and metabolism.

In conventional vagus nerve stimulation, a device is surgically implanted under the skin of a subject's chest, and a wire is threaded under the skin connecting the device to the left cervical vagus nerve. When activated, the device sends electrical signals along the left vagus nerve to the brainstem, which then sends signals to certain areas in the brain. Conventionally, the right vagus nerve is not used because it can be more likely to carry fibers that supply nerves to the heart. However, the right vagus also contains the dominant parasympathetic fibers innervating the gut and especially the liver; thus, unlike conventional vagus nerve stimulation and previously used configurations, the stimulation described herein is placed on the right cervical vagus nerve or the subdiaphragmatic common hepatic branch. This subdiaphragmatic branch of the vagus nerve does not contain fibers that extend to the heart but does have connections to the liver and other gastrointestinal organs, and through testing related to the present disclosure has been shown to produce meaningful changes in urea cycle enzymes.

However, despite the foregoing, the complex relationship between the ANS and whole-body metabolism remains elusive. For instance, although a decrease in blood glucose levels after vagal nerve stimulation has been documented in preclinical settings, cervical vagal nerve stimulation seems to impair insulin release. This inconsistency might be partly attributed to the differences in afferent and efferent vagal nerve stimulation but can also be explained by mixed endocrine and vagal signaling to the liver. Importantly, these discrepancies highlight the unpredictable nature of the ANS and the need to explore and better understand the role of the ANS in the regulation of systemic metabolism in organ- and context-specific manners.

Data provided in the examples below shows that denervation or stimulation of the vagus nerve impacts the urea cycle in the liver. In embodiments, stimulating the parasympathetic nervous system comprises stimulating the vagus nerve. For example, stimulating the vagus nerve can comprise stimulating the cervical vagus nerve or the hepatic branch of the vagus nerve.

The Examples provided below show that vagus nerve manipulation can affect liver metabolism and help restore systemic nitrogen homeostasis in subjects having cancer associated cachexia. The examples include investigations of systemic- and liver-specific nitrogen-related changes during cancer associated cachexia (Example 1) and ANS perturbation (Example 2) and evaluation of the hypothesis that ANS intervention can mitigate or reverse nitrogen and urea cycle dysregulation during cachexia (Example 3). Additional examples evaluate the impact of ANS intervention in subject's having cachexia on weight loss, body fat (total and brown adipose tissue), muscle mass, and food intake (Example 4).

The mouse models used in the examples were established KPC and LLC models, which are representative models for the study of cancer cachexia that robustly recapitulate features of human disease. The KPC model is described fully in Michaelis et al., Establishment and characterization of a novel murine model of pancreatic cancer cachexia (“Michaelis”), which is incorporated by reference herein. Briefly, Michaelis describes that syngeneic KPC allografts are a robust model for studying cachexia, which recapitulate key features of the pancreatic ductal adenocarcinoma (PDAC) disease process and induce a wide array of cachexia manifestations. As such, this model is ideally suited for future studies exploring the physiological systems involved in cachexia and for preclinical studies of novel therapies. The LLC model is described fully in Choi, et al., Concurrent muscle and bone deterioration in a murine model of cancer cachexia (“Choi”), which is incorporated by reference herein. Briefly, Choi describes testing the validity of the Lewis Jung carcinoma (LLC) as a model of cancer cachexia and examining its effect on the two major lean tissue components, skeletal muscle, and bone. Choi concluded that LLC is a valid model of cachexia that induces rapid losses in global bone mineral density and in limb and respiratory muscle function. Both KPC and LLC models combine established spontaneously occurring animal models and genetically engineered mouse models to ensure conservation of pathways across a diverse array of cancers.

Vagus nerve manipulation includes stimulation of the parasympathetic vagus nerve, including areas of the parasympathetic vagus nerve such as the cervical vagus branch or the hepatic vagus nerve branch.

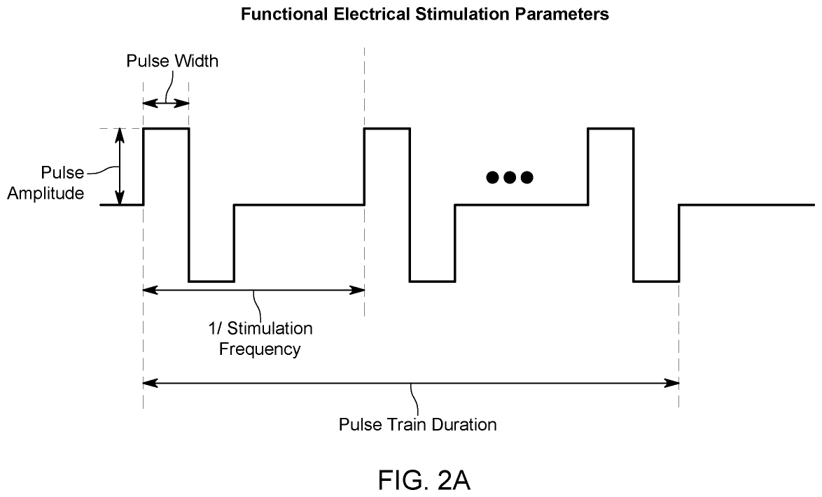

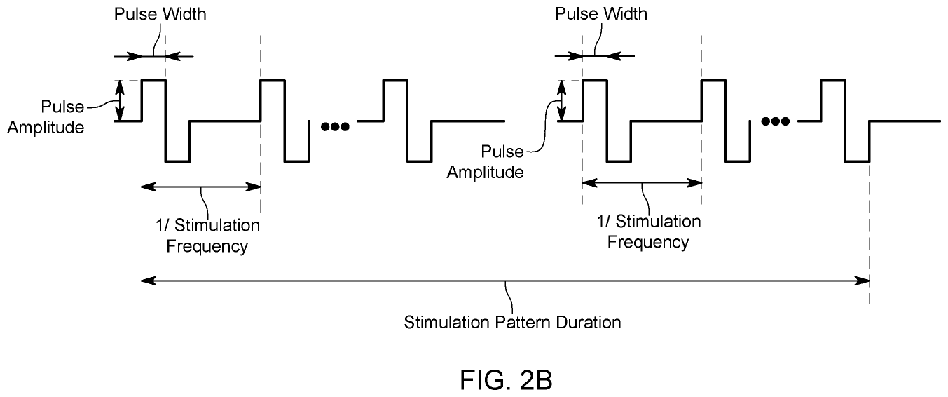

In functional electrical stimulation, typically positive or negative pulsed current is delivered from electrode surface contacts at the peripheral nerve location. This methodology is generally considered to be more physiologically relevant and produces less damage than sending a continuous signal. FIG. 2A provides an exemplary schematic diagram of functional electrical stimulation parameters. Pulse amplitude is variable and is generally titrated for an individual, but pulse width and stimulation frequency are set based on neurophysiology principles regarding evoking or suppressing activity along the vagus nerve. The pulse width, frequency, and amplitude are factors that can be used to describe an electrical stimulus. Additional parameters that can be used to describe an electrical stimulus include pulse train duration and stimulation pattern duration. Pulse train duration defines the amount of time a pulse train continues over a period of time. Stimulation pattern duration is the time duration a pulse train repeats over a period of time. FIG. 2B provides an exemplary schematic diagram of stimulation pattern duration.

Different stimulation frequencies may modulate nerves differently. For example, 10 Hz pulses are commonly used to activate peripheral nerves, while high-frequency pulses (e.g., 5 kHz) have been shown to block peripheral nerves.

In embodiments, the frequency of the pulses delivered to the vagus nerve includes frequencies ranging from 1 Hz to 10 Hz. For example, the frequency may be 1 Hz, 2 Hz, 3 Hz, 4 Hz, 5 Hz, 6 Hz, 7 Hz, 8 Hz, 9 Hz, or 10 Hz. In this frequency range, a pulse may have a pulse width of about 1 millisecond to about 100 milliseconds. For example, the pulse width may be about 1 millisecond (ms) to about 10 ms, including about 1 ms, 2 ms, 3 ms, 4 ms, 5 ms, 6 ms, 7 ms, 8 ms, 9 ms, or 10 ms. The pulse may be a charge balanced constant current biphasic pulse with alternating anodic and cathodic leading phases with a range of 0.1 mA to 10 mA.

In other embodiments, the frequency of the pulses delivered to the vagus nerve includes a frequency of 5 kHz. At or near this frequency range, a pulse may have a pulse width of about greater than 0 and less than or equal to 0.2 milliseconds. For example, the pulse width may be about 0.5 ms, 1 ms, 1.5 ms or 2 ms. The pulse may be a charge balanced constant current biphasic pulse with alternating anodic and cathodic leading phases with a range of 0.1 mA to 10 mA.

In embodiments, pulse train duration and stimulation pattern duration can vary over a range values and can be affected by other stimulation parameters, such as, for example, pulse width, frequency, and amplitude. For example, the pulse train duration can range from 1 minute to 1 hour, and the stimulation train duration can vary from 20 minutes to 3 hours.

The following Examples are provided by way of illustration and not by way of limitation.

### EXAMPLES

### Example 1. Characterization of the Systemic Nitrogen-Associated Metabolic Changes in the Host During CAC

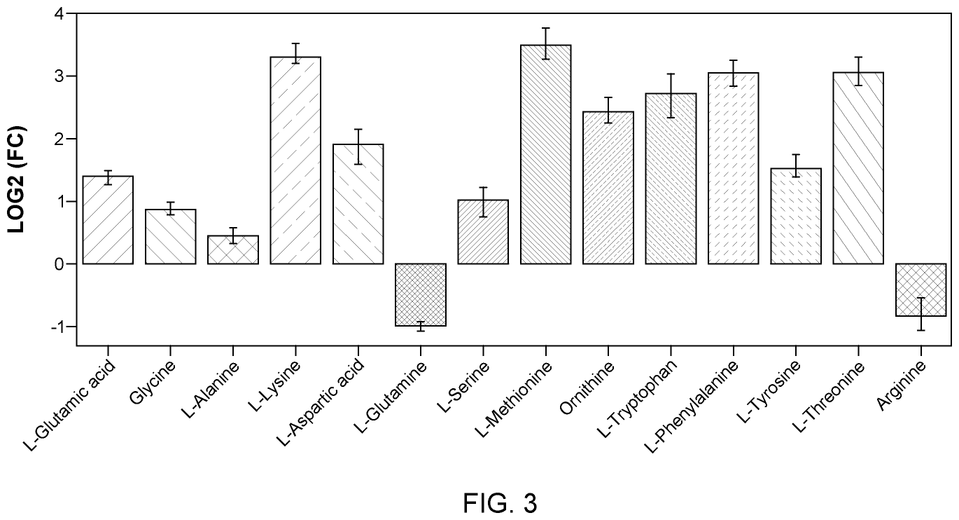

Amino acid levels in cachectic liver. Amino acid levels in livers from tumor-bearing cachectic mice and control mice were measured and compared. Mice were injected with saline to produce healthy control mice, or a cancer cell line known to produce robust cachexic phenotypes, including Lewis lung carcinoma (LLC) or KRAS, p53, and Cre pancreatic cancer (KPC). Using liquid chromatography mass spectrometry (LC-MS) metabolomics on urine, plasma, and homogenates of excised liver, muscle, and tumors, significant alterations in hepatic amino acid levels in cachectic mice were observed compared to control mice. FIG. 2 is a graph showing the fold-changes of hepatic amino acid levels in cachectic mice using Student's t-test, Bonferroni FDR<0.05. FIG. 3 graphically illustrates the results. As can be seen, the LC-MS analysis showed fold-change decreases in glutamine and arginine and increases in ornithine and aspartic acid for the cachectic subjects versus control, which supports the idea that cachexia causes urea cycle dysfunction.

### Example 2. Characterization of the Effects of Hepatic Sympathetic and Parasympathetic Interventions on the UC

Testing was performed to evaluate whether the autonomic nervous system plays a role in modulating the hepatic urea cycle. Denervation and electrical stimulation of the hepatic vagus nerve were performed to manipulate the ANS. Briefly, microwire cuff electrodes were placed on the cervical or subdiaphragmatic vagus nerve to enable VNS. The inventor performed the same metabolic and transcriptomic profiling described in Example 1 to measure the impact of ANS perturbation on liver metabolism. Briefly, LC-MS based metabolomics and RNA-seq were carried out on the harvested liver cells to measure the metabolite and enzyme expression levels. The inventor also performed 15N-labeled nitrogen tracing following the ANS interventions.

Denervation surgery. All mice were anesthetized by intraperitoneal (i.p.) injection of a mix of ketamine-medetomidine-atropine (KMA) and after the surgery received a subcutaneous (s.c.) injection of antisedan and buprenorphine. The abdomen was shaved and sterilized using alternating applications of ethanol and iodine.

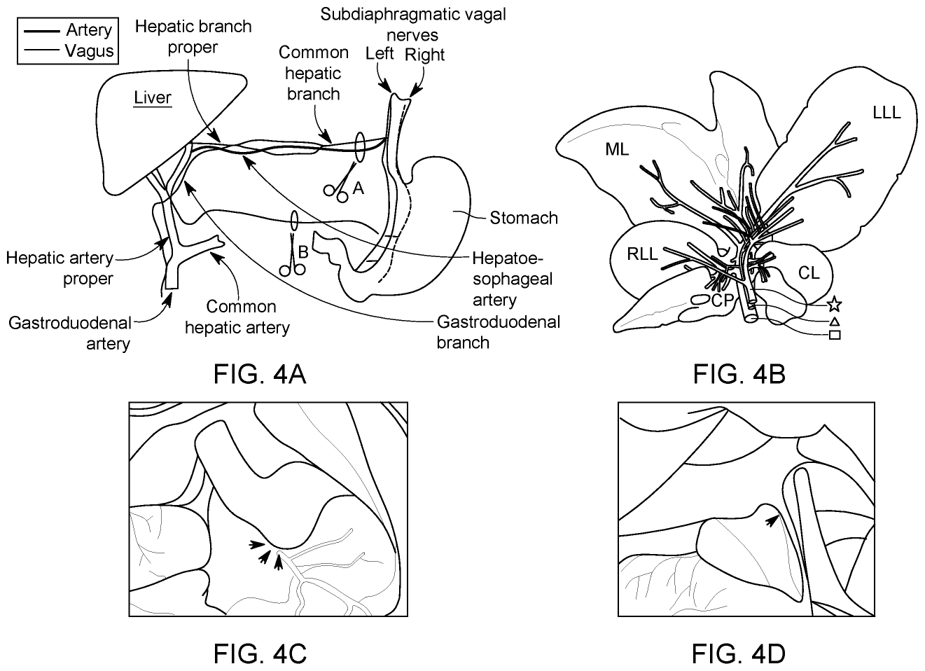

FIGS. 4A-4D provide schematic illustrations and photographic images showing denervation surgery. FIG. 4A is a schematic representation illustrating the positions of the subdiaphragmatic vagal nerves. In FIG. 4A, the upper branch, labeled (A) is dissected to achieve a hepatic parasympathectomy, and the lower branch labeled (B) is severed to achieve a hepatic sympathectomy. FIG. 4B is an illustration of the underside of the medial and left lobes of the liver showing the hepatic artery (triangle), portal vein (square), and bile duct (star). Nerve bundles running along the hepatic artery were transected for a hepatic sympathectomy. FIG. 4C is an image taken during surgery of the sympathetic hepatic nerve branches entering the liver with the left and medial lobes lifted to expose the major vascular and biliary tracts. Nerve bundles, corresponding to those needed to be severed for a sympathectomy, are marked with arrows. FIG. 4D is an image taken during surgery of the left subdiaphragmatic vagal nerve running along the esophagus. The common hepatic vagus nerve branch is marked with an arrow. Severing this connection results in a parasympathectomy.

A ventral vertical midline incision was obtained by cutting the skin and muscle layers to visualize the liver. A binocular operating microscope was used for the remainder of the surgery. For the hepatic sympathectomy (Sx), the median and left liver lobes were lifted to visualize the region comprising the bile duct, hepatic artery, and portal vein (FIG. 4B). Nerve bundles running along the hepatic artery proper were transected using microsurgical instruments. Any connective tissue attachments between the hepatic artery, bile duct, and portal vein were dissected (FIG. 4C). To achieve a hepatic parasympathectomy (Px), the common hepatic vagal branch was transected by stretching the fascia containing the common hepatic vagal branch and transecting neural tissue between the ventral vagal trunk and liver (FIG. 4D). In mice with a sham denervation surgery, the same procedure of incision and nerve exposure was performed, but the nerves were not severed. After the denervation, warm saline was injected into the abdominal cavity, and the muscle and skin layers were stitched.

Electrode Implantation: All mice were anaesthetized by intraperitoneal (i.p.) injection of ketamine-medetomidine-atropine (KMA) and after the surgery receive a subcutaneous (s.c.) injection of antisedan and buprenorphine. The abdomen was shaved and sterilized using alternating applications of ethanol and iodine. A ventral vertical midline incision was performed by cutting the skin and muscle layers to visualize the liver. A binocular operating microscope was used for the remainder of the surgery. Medtronic Streamline unipolar myocardial pacing leads were implanted for stimulation and recording (FIG. 4A).

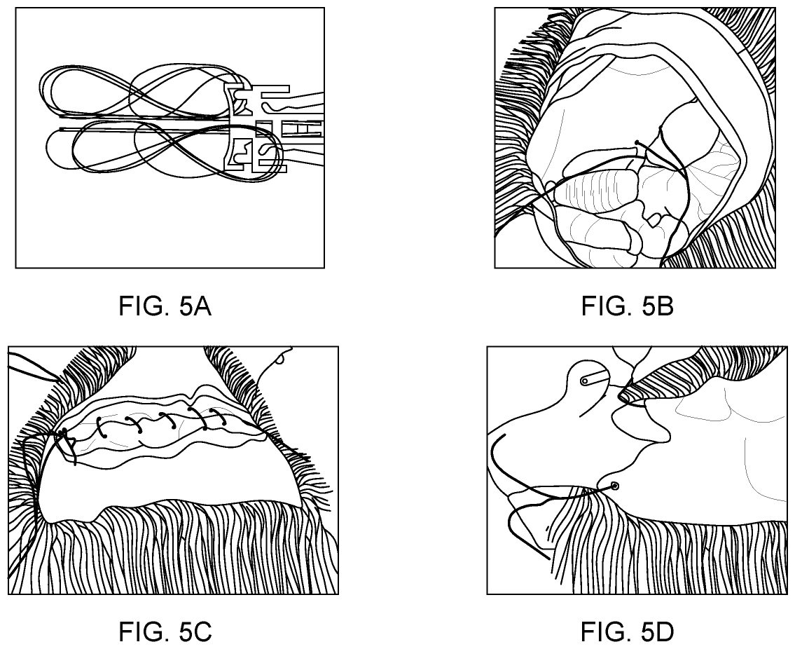

FIGS. 5A-5D provide images showing electrical stimulation of hepatic sympathetic and parasympathetic nerves. FIG. 5A is a photograph of clinical pacemaker leads for stimulus and recording. FIG. 5B is a photograph of leads implanted for recording at the hepatic sympathetic nerve branch (triangle) and stimulation at the hepatic parasympathetic nerve branch (star). FIG. 5C is a photograph of the abdominal muscle closed with stitches, with leads exiting the incision site; leads were tunneled subcutaneously along the back before the skin was closed. FIG. 5D is a photograph of leads exiting the subcutaneous space at the neck, which are kept from re-entering with a loose knot in the lead.

The recording electrode coil was implanted around the parasympathetic branch of the hepatic nerve, and the stimulation recording electrode coil was implanted around the sympathetic hepatic nerve (FIG. 5B). The tailing ends of the leads were tunneled s.c. and exited at the base of the neck to prevent the animal from chewing on the wires (FIGS. 5C, 5D). Finally, warm saline was injected into the abdominal cavity, and the muscle and skin layers were stitched.

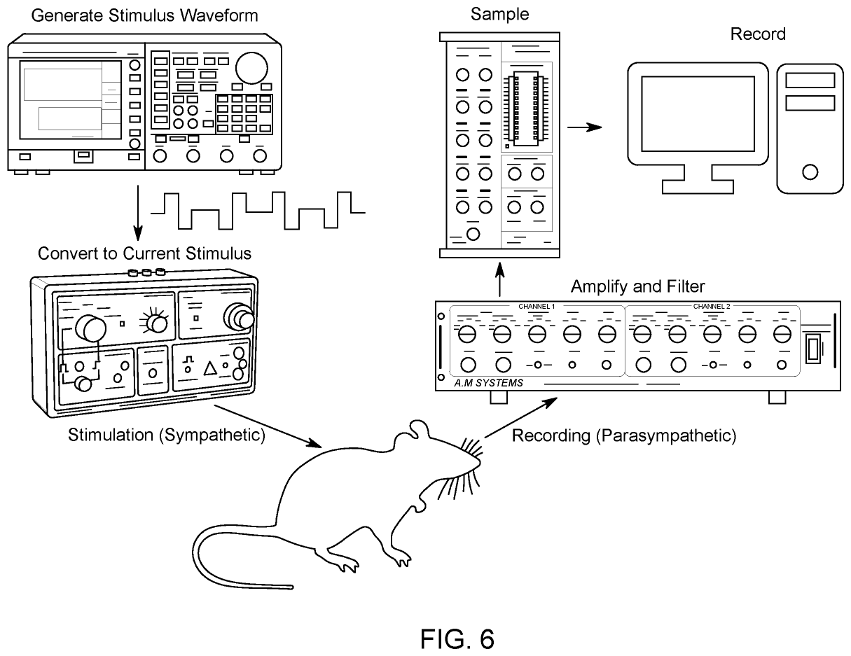

Electrical Stimulation and Recording. The stimulus applied comprised charge balanced 2 ms biphasic pulses delivered at 10 Hz with alternating anodic and cathodic leading phases of amplitude 0.1 to 10 mA, generated via a Tektronix AFG1062 arbitrary function generator and converted via a Digitimer DS5 isolated bipolar current stimulator. Recordings were amplified 100× and isolated using a 300-5,000 Hz bandpass filter via an A-M Systems microelectrode AC amplifier model 1800 and recorded using a National Instruments BNC-2110 with a 10 kHz sampling rate (FIG. 6).

FIG. 6 is a schematic illustration showing a block diagram outlining the stimulation and recording pipeline used in the present example.

Preliminary data on ANS stimulation: The effect of sympathetic and parasympathetic electrostimulation on glucose was evaluated. To test the impact of sympathetic and parasympathetic nervous system stimulation on metabolism, animals were implanted with stimulation devices for sympathetic nerve (N=3), parasympathetic nerve (N=3), or sham (N=2) stimulation. For the testing, blood glucose levels were assessed using a glucose meter and a single drop of blood. Measurements were taken before, during, and after stimulation. Stimulation was delivered as described in the previous section to awake, unanesthetized animals. For glucose tolerance tests, stimulation was delivered for 45 minutes, with glucose challenge injected intraperitoneally at 15 minutes into the study. For fasting glucose tests, baseline blood glucose measurements were taken for ten minutes before stimulation was initiated. Stimulation was delivered for 30 minutes, during which blood glucose levels were also evaluated. Blood glucose levels were also assessed for 100 minutes post-stimulation.

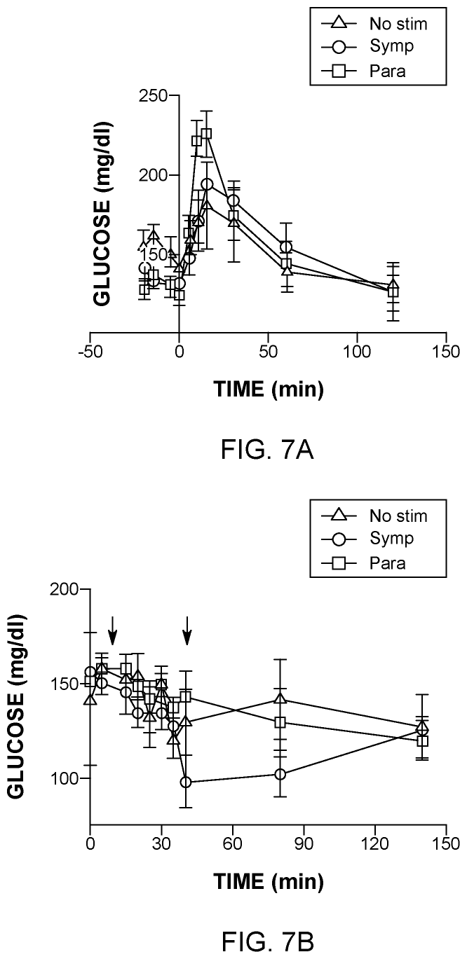

FIGS. 7A-7B are graphs showing blood glucose level after sympathetic (Symp), parasympathetic (Para) and sham (No stim) stimulations in the present example. For the results shown in FIG. 7A, an intraperitoneal glucose tolerance test with nerve stimulation from time—15 to 30 min was performed, and glucose was injected at time 0 min. For the results shown in FIG. 7B, the effect of nerve stimulation on fasting blood glucose was evaluated. The nerves were stimulated from time 10 to 40 min. The testing showed that parasympathetic stimulation impaired glucose tolerance upon i.p. glucose challenge compared to the sham control whereas sympathetic stimulation did not (FIG. 7A). The blood glucose level returned to baseline at a similar time for all groups. Conversely, sympathetic stimulation significantly lowered fasting blood glucose levels even after its cessation while parasympathetic stimulation did not (FIG. 7B). This experiment suggests that the whole-body glucose metabolism can be modulated by the ANS-mediated gut-brain axis.

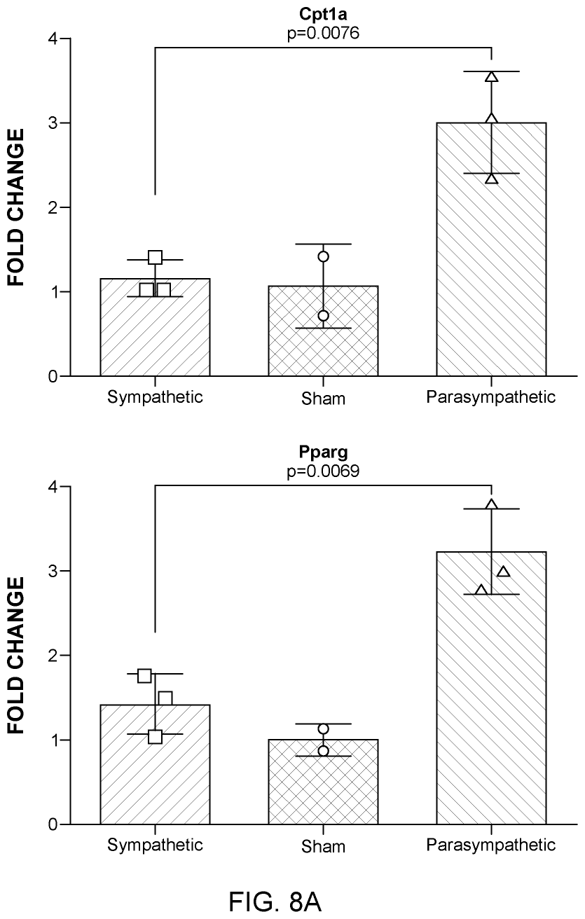

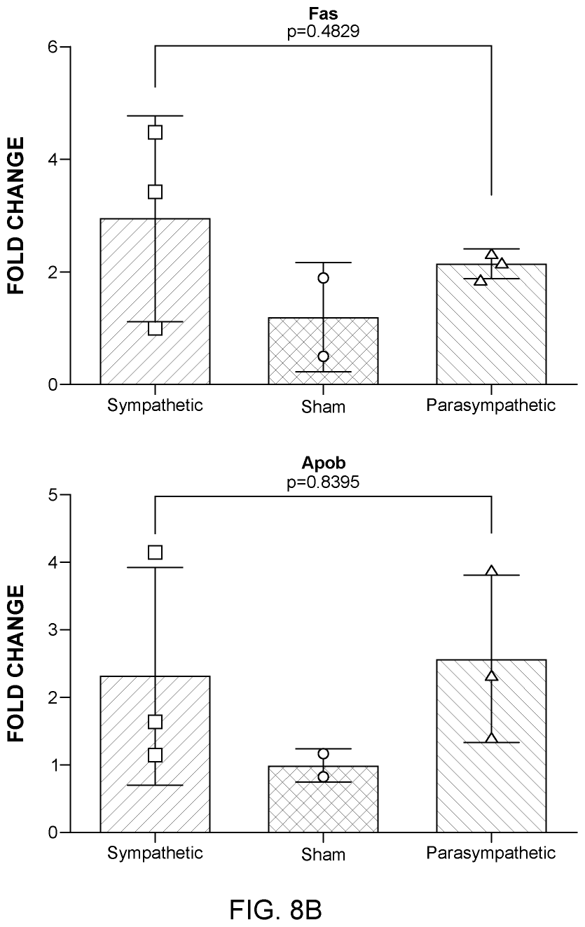

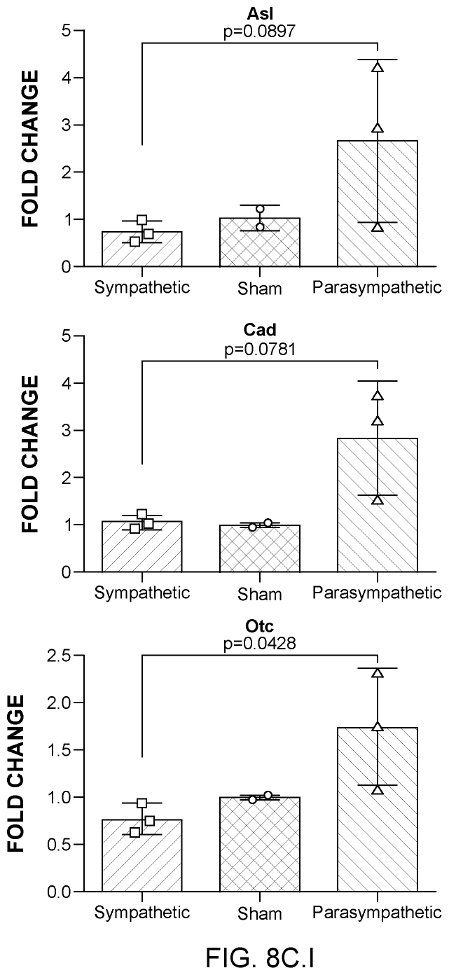

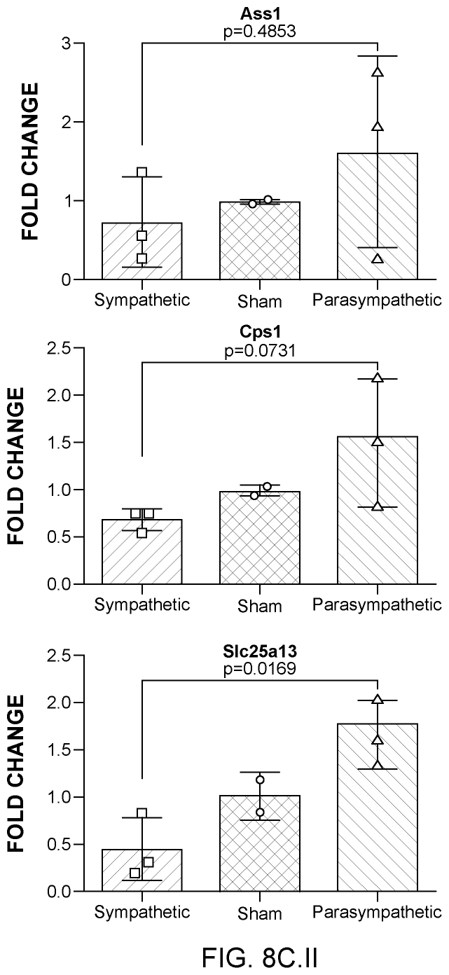

FIGS. 8A-8C are graphs showing hepatic metabolic gene expression levels quantified by qRCR after sympathetic and parasympathetic stimulation in this example. FIG. 8A shows expression of lipid catabolismgenes. FIG. 8B shows expression of lipogenesis and VLDL genes. FIG. 8C shows expression of urea cycle enzymes. Sham, N=2. Sympathetic stimulation, N=3. Parasympathetic stimulation, N=3. Error bar, ±SD.

The results suggest that ANS stimulation modulates hepatic lipid and urea cycle metabolism. Despite the limited number of animals in the preliminary study, parasympathetic stimulation alone increased lipid catabolism-related gene expression in the liver (FIG. 8A), while sympathetic and parasympathetic stimulation similarly increased lipogenesis- and VLDL-related gene expression (FIG. 8B).

Parasympathetic stimulation generally increased expression of urea cycle enzymes in the liver more than sympathetic stimulation (FIG. 8C). Therefore, based on the data shown in the figures, stimulation of the parasympathetic nervous system has the potential to increase urea cycle flux, which may be able to reverse or mitigate cachectic urea cycle dysregulation.

Together, these preliminary results suggest that ANS regulates liver metabolism in general and specifically expression of genes related to hepatic urea cycle.

### Example 3. ANS Interventions Reverse and/or Mitigate UCD and Cachectic Phenotypes

Cancer cachexia is defined by progressive weight loss greater than 5% or 2% in individuals already showing decrease in BMI or depletion in skeletal muscle mass that cannot be fully reversed by conventional nutritional support. As demonstrated in Example 1, the inventor was able to recapitulate this phenotype using the KPC pancreatic and LLC lung mouse cancer models. All injected mice developed pancreatic cancer in the pancreas, and there was no correlation between mouse weight at injection time and survival (data not shown). Testing in the cachectic phenotype of the KPC pancreatic cancer mouse model showed a prominent change in muscle mass in addition to the described loss of fat mass (data not shown).

To rule out potential contribution to nitrogen dysregulated homeostasis by the microbiome that may confound data interpretation, the KPC mice were challenged with broad-spectrum antibiotic treatment. Weight was tracked in four groups: mice receiving antibiotics with or without KPC, mice not receiving antibiotics and without KPC, and mice with KPC not receiving antibiotics. The results showed that mice with KPC lost weight; however, antibiotic treatment did not have a significant effect on the magnitude of weight loss or on survival (data not shown). Thus, the results showed that the microbiome did not contribute to nitrogen dysregulated homeostasis.

ANS intervention to mitigate cachexia. ANS intervention was evaluated for ability to reverse hepatic urea cycle dysregulation and mitigate the cachexia phenotype. The following experiments were performed:

1. Hepatic sympathetic and parasympathetic denervation. The first experiment examined (a) whether hepatic sympathetic or parasympathetic nerve activities contributed to the onset and progression of cachexia and (b) whether denervation could prevent or deter cachexia. As described in Example 2, hepatic sympathetic and parasympathetic denervation, as well as sham surgeries, were performed (see FIG. 4). Following surgery, the animals were allowed to recover for ˜1 week prior to implantation with tumor cells for the KPC and LLC cachectic models. Tumor burden, weight loss, and survival were measured during disease progression.

2. Hepatic sympathetic and parasympathetic stimulation. An evaluation of whether neurostimulation can reverse or mitigate cachectic phenotypes was performed. As described in Example 2, the electrodes were implanted to stimulate the hepatic sympathetic or parasympathetic nerves (FIG. 3). The animals were allowed to recover for ˜1 week before KPC or LLC tumor cells were injected. After detection of cachexia based on the diagnostic criterion (weight loss greater than 5% or weight loss greater than 2% with BMI<20 kg/m2 with depletion in skeletal muscle mass), which is recapitulated by the KPC pancreatic and LLC lung mouse cancer models), daily stimulation of the sympathetic and parasympathetic nerves using 10 Hz (activation), 4 KHz (blocking), and sham (control) were performed. Tumor burden, weight loss, and survival were monitored.

Preliminary data. An evaluation of whether perturbation of the sympathetic system has an independent effect on mice with cancer was performed. The sympathetic system was denervated using the norepinephrine analog 6-hydroxydopamine (6-OHDA) in mice injected with lung cancer cells. Post sacrifice, the RNA levels of urea cycle enzymes in the livers were analyzed. Sympathetic denervation was found to decrease the expression of urea cycle enzymes (unpublished data).

### Example 4

Sympathetic and parasympathetic stimulations using biphasic pulses delivered at 5 kHz were evaluated.

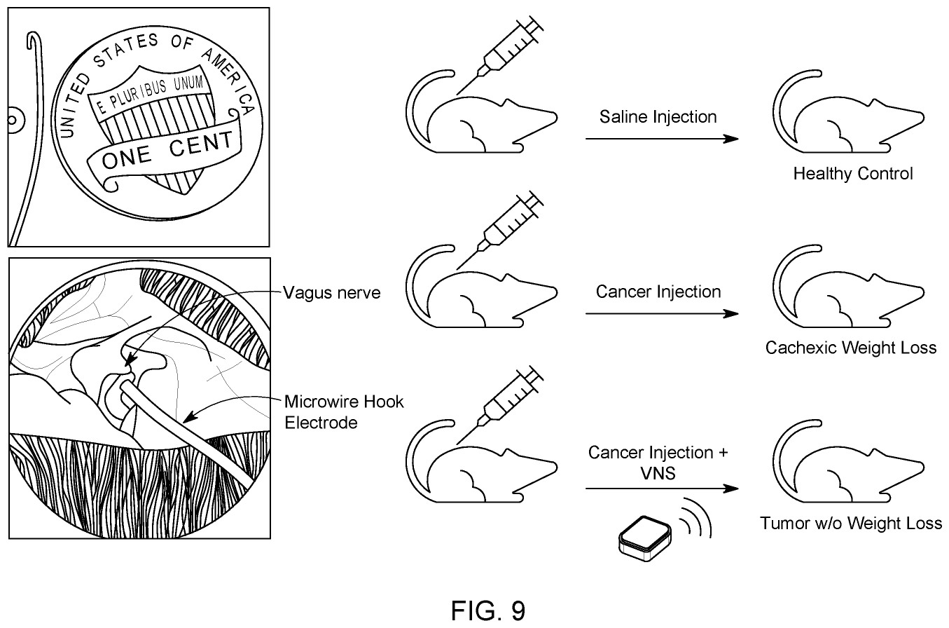

FIG. 9 provides photographs and a schematic illustration of experimental procedures for Example 4. The top left photograph shows an exemplary microwire hook electrode used in the procedures with a penny for scale. As shown in the bottom left photograph, the same electrode was implanted on the right cervical vagus nerve of a C57B6/J mouse to deliver vagus nerve stimulation. Electrode lead wires were tunneled subcutaneously to the back of the neck, where a transcutaneous port for connections to the stimulation device was employed. Post-implantation, mice were given a recovery window of 3-7 days post-surgery before treatment began. Mice were randomly assigned to either healthy control (saline injection), cancer with no therapy, or cancer with vagus nerve stimulation therapy.

Cancer mice were injected with established cancer cell lines for studies of cancer cachexia, including KPC and LLC. Therapeutic stimulation was delivered daily to the cervical vagus nerve for 30 minutes at the beginning of the wake cycle. Stimulation comprised charge balanced biphasic stimulation delivered at 5 Hz with 100 ms pulse width and 50-300 mA amplitude as titrated to produce local muscle twitch and up to 10% change in heartrate. Mice were evaluated daily for weight loss, tumor burden, and food intake. At study termination, they were additionally assessed for body fat content, muscle integrity, and other physiologic hallmarks of cachexic phenotypes and mechanisms. The results of these studies are outlined in the following paragraphs.

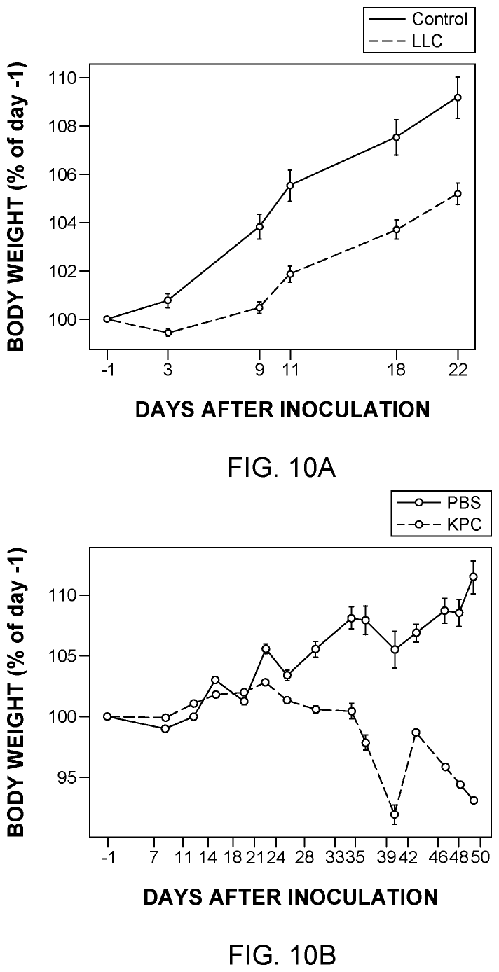

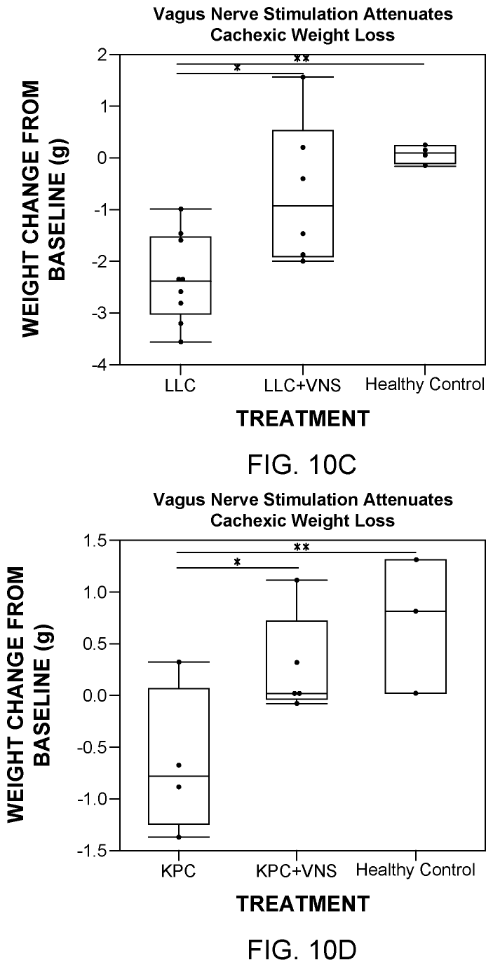

The effect of cancer xenograft on weight loss for mice that were both treated and untreated with VNS was evaluated. FIGS. 10A-10D are charts and graphs illustrating how body weight was affected by cancer injection and vagus nerve stimulation. FIG. 10A shows change in body weight for mice inoculated with LLC. FIG. 10B shows change in body weight in mice inoculated with KPC. The cancer cell lines produced a marked cachexic phenotype, including marked weight loss compared to healthy controls. FIGS. 10C and 10D show change in body weight for mice treated with vagus nerve stimulation therapy. FIG. 10C shows results for mice inoculated with LLC and FIG. 10D shows results for mice inoculated with KPD. As can be seen, VNS therapy provided a statistically significant reduction in cachexic weight loss compared to untreated cancer mice, without statistically significant variation from healthy control animals as evaluated at humane endpoints for the study. *: p<0.05 **p<0.01 ***p<0.001.

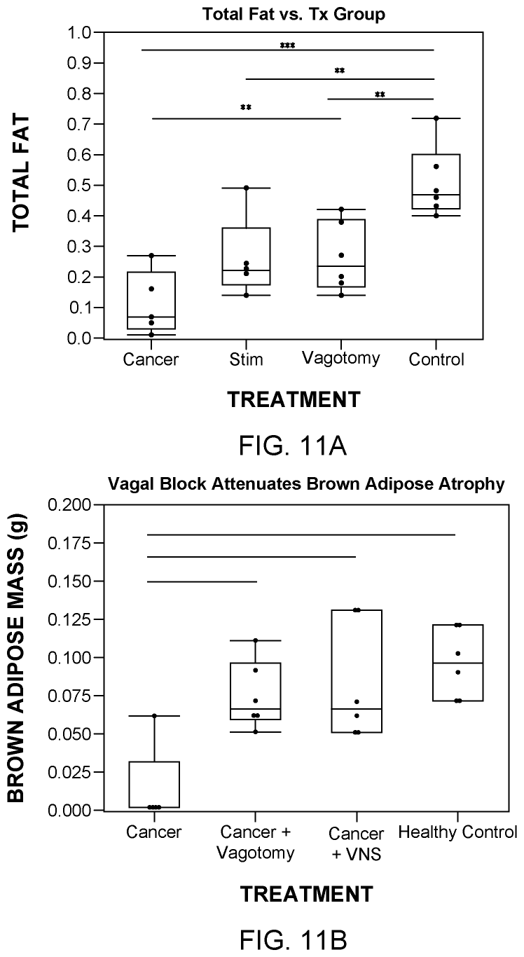

The effect of cancer xenograft on total fat and brown adipose tissue was evaluated for cancer mice that both received and did not receive VNS. FIGS. 11A and 11B are charts showing the effect of VNS or the absence of VNS on total fat and brown adipose tissue, respectively. FIG. 11A illustrates that VNS, as well as vagal purturbation through right cervical vagotomy, provide attenuation of fat loss. FIG. 11B illustrates that brown adipose tissue (which is a type of fat that is critical for maintaining homeostasis and normal metabolic function) atrophy was pronounced in cachexic animals, but there was no significant change in BAT observed between healthy controls and cancer animals that received VNS or vagotomy therapies. The quantification were made at humane endpoints for the study (approximately 2 weeks post-inoculation). *: p<0.05 **p<0.01 ***p<0.001.

Another clinical feature that impacts quality of life for cachexic patients is muscle wasting and loss of skeletal muscle mass. A common quantitative approach to clinical assessment of muscle loss is quantification of mean muscle fiber diameter from patient biopsies, as the force generation a muscle is capable of is directly proportional to the size of the muscle fibers. Thus, muscle biopsys were used to quantify mean muscle fiber diameter from mice in the study.

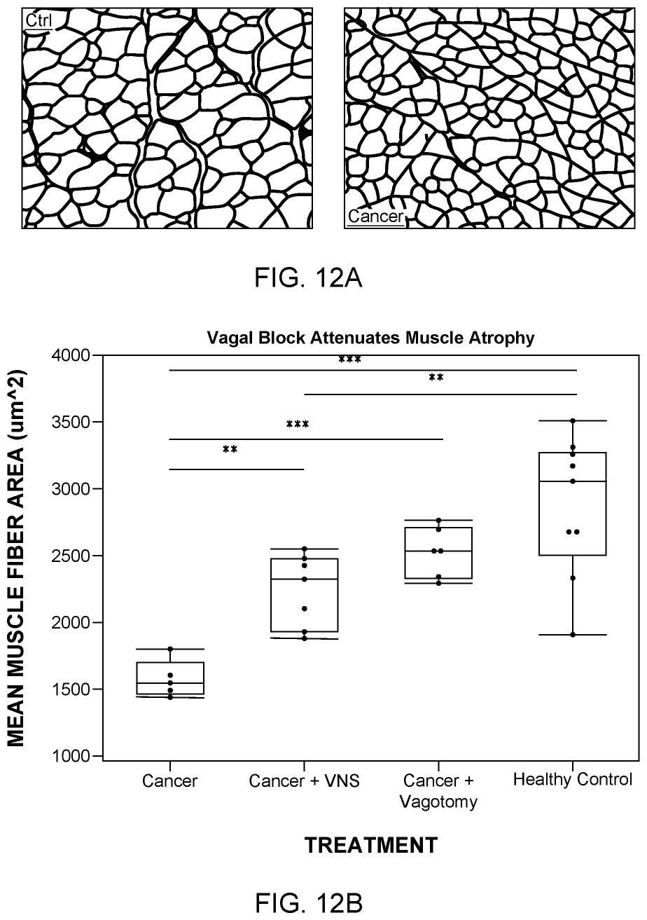

At study termination, muscle was collected from the left thigh and assayed, using a wheat germ agglutinin stain to visualize cross-sectional area of skeletal muscle fibers (left). FIG. 12A includes photographs of skeletal muscle fiber for Control (top) and Cancer (bottom) mice. FIG. 12B is a chart comparing muscle atrophy for mice having cancer, cancer with VNS therapy, cancer with vagotomy, and healthy control. As shown in FIG. 12B, VNS therapy significantly attenuated the atrophy of muscle fibers compared to cachexic mice receiving no treatment, as evidenced by less reduction in muscle fiber size compared to controls. *: p<0.05 **p<0.01 ***p<0.001.

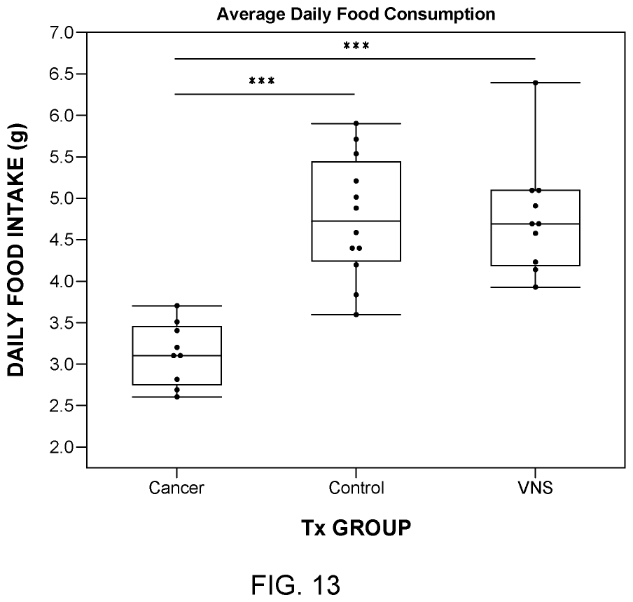

Another hallmark of cachexia, which has a pronounced impact on patient quality of life, is anorexia. While dietary intake alone is insufficient to explain cachexia (high fat, high protein, and high calorie diets have not proved effective tools clinically for cachexia treatment, nor has tube feeding), patients routinely report reduced appetite as part of the complex metabolic syndrome and systemic symptoms. FIG. 13 is a chart showing the effect of vagal nerve stimulation on daily food intake for control mice, mice with cancer but no treatment, and cancer with VNS treatment. As shown, cachexic mice have significantly reduced food intake despite having free access to unlimited food, which is reversed in the cohort receiving vagal perturbation therapy. *: p<0.05 **p<0.01 ***p<0.001. As shown, there is no statistical difference between the control group and the cancer group having vagus nerve stimulation therapy.

One skilled in the art will readily appreciate that the present disclosure is adapted to carry out the objects and obtain the ends and advantages mentioned, as well as those inherent therein. The present disclosure described herein is representative of exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method for treating cachexia in a subject in need thereof, the method comprising stimulating the parasympathetic nervous system of the subject thereby treating cachexia in the subject.

2. The method of claim 1, wherein stimulating the parasympathetic nervous system in the subject increases expression of urea cycle enzymes in the liver.

3. A method for mitigating weight loss due to cachexia in a subject in need thereof, the method comprising stimulating the parasympathetic nervous system in the subject thereby mitigating weight loss due to cachexia.

4. The method of claim 4, wherein the subject having cachexia who received stimulation to the parasympathetic nervous system experiences statistically significantly less weight loss than subjects having cachexia that received no stimulation.

5. The method of claim 4, wherein there is no statistically significant variation in weight loss between the subject having cachexia who received stimulation to the parasympathetic nervous system and a healthy control subject.

6. A method for mitigating fat loss due to cachexia in a subject in need thereof, the method comprising stimulating the parasympathetic nervous system in the subject thereby mitigating fat loss due to cachexia.

7. The method of claim 6, wherein the fat is brown adipose tissue.

8. The method of claim 7, wherein there is no significant change in brown adipose tissue mass between healthy control subjects and subjects having cachexia who received stimulation to the parasympathetic nervous system.

9. The method of claim 6, wherein subjects having cachexia who received stimulation to the parasympathetic nervous system experience statistically significantly less atrophy of brown adipose tissue than subjects having cachexia that received no stimulation.

10. A method for mitigating muscle wasting due to cachexia in a subject in need thereof, the method comprising stimulating the parasympathetic nervous system in the subject thereby mitigating muscle wasting due to cachexia.

11. The method of claim 10, wherein subjects having cachexia who received stimulation to the parasympathetic nervous system experience statistically significantly less muscle atrophy than subjects having cachexia that received no stimulation.

12. A method for mitigating loss of appetite due to cachexia in a subject in need thereof, the method comprising stimulating the parasympathetic nervous system in the subject thereby mitigating loss of appetite.

13. The method of claim 12, wherein there is no statistically significant variation in average daily food intake between subjects having cachexia who received stimulation to the parasympathetic nervous system and healthy control subjects.

14. The method of claim 12, wherein subjects having cachexia who received stimulation to the parasympathetic nervous system had statistically significantly higher daily food intake than subjects having cachexia that received no stimulation.

15. A method for mitigating urea cycle dysregulation in a subject in need thereof due to cachexia, the method comprising stimulating the parasympathetic nervous system in the subject thereby mitigating urea cycle dysregulation due to cachexia in the subject.

16. The method of claim 1, wherein stimulating the parasympathetic nervous system comprises stimulating the vagus nerve.

17. The method of claim 16, wherein stimulating comprises stimulating the cervical vagus nerve or the hepatic branch of the vagus nerve.

18. The method of claim 16, wherein stimulating comprises electrical stimulation or optogenetic stimulation.

19. The method of claim 16, wherein stimulating the vagus nerve comprises delivering electrical pulses at a frequency ranging from 1 Hz to 10 Hz.

20. The method of claim 19, wherein the pulses have a pulse width of about 1 millisecond to about 100 milliseconds.

21. The method of claim 20, wherein the pulses have a pulse width of about 1 millisecond to about 10 milliseconds.

22. The method of claim 19, wherein the pulses are delivered at a frequency from 5 Hz to 10 Hz.

23. The method of claim 19, wherein stimulating the vagus nerve comprises delivering charge balanced constant current biphasic pulses with alternating anodic and cathodic leading phases with a range of 0.1 mA to 10 mA.

24. The method of claim 16, wherein stimulating the vagus nerve comprises delivering electrical pulses at a frequency of about 5 kHz.

25. The method of claim 24, wherein the pulses have a pulse width of greater than 0 and less than 0.2 milliseconds.

26. The method of claim 16, wherein stimulating the vagus nerve comprises delivering electrical pulses having a pulse train duration ranging from 1 minute to 1 hour.

27. The method of claim 16, wherein stimulating the vagus nerve comprises delivering electrical pulses having a stimulation train duration ranging from 20 minutes to 3 hours.

28. The method of claim 24, wherein stimulating the vagus nerve comprises delivering biphasic pulses with alternating anodic and cathodic leading phases with a range of 0.1 mA to 10 mA.

29. The method of claim 1, wherein the cachexia is cancer-associated cachexia.

30. The method of claim 29, wherein the associated cancer comprises pancreatic cancer or lung cancer.
